(19) United States
(12) Patent Application Publication
(10) Pub. No.: US 2009/0234315 A1
(43) Pub. Date: Sep. 17, 2009

(54) Disposable nursing pad with non-slippage backing

(76) Inventor: Rosa G. Gomez, Mint Hill, NC (US)

Correspondence Address: Jerome J. Norris, SUITE 305, 1901 PENNSYLVANIA VE., N.W., WASHINGTON, DC, 20006, US

(21) Appl. No.: 12/292,425

(22) Filed: Nov. 19, 2008

Related U.S. Application Data
(60) Provisional application No. 61/003,579, filed on Nov. 19, 2007.

(51) Int. Cl. A61F 13/15; A41C 3/00

(52) U.S. Cl. 604/385.07; 450/37; 450/81

(57) ABSTRACT

A disposable breast pad containing a multi-functional pressure sensitive adhesive to hold the breast pad and adjoining bra firmly in place without slippage upon leakage of fluids from the breast of nursing or pregnant human females, comprising:
a) outer peripheral circumference layer means disposed on a women's breast;
b) inner absorbent layer means disposed on an inner section of the outer layer means in contact with the breast to absorb leaking secreted fluids; wherein the inner absorbent layer is a multi-functional pressure sensitive adhesive that is moisture absorbing, and moisture transmitting.

### BACKGROUND OF THE INVENTION

The present invention relates to a disposable breast pad for nursing and pregnant human females who often times have unexpected leakage of milk from the breast and claims the benefit of the Nov. 19, 2007 filing date of provisional application No. 61/003,579. In particular, the disposable nursing pad with non-slippage backing has an inner portion which is in direct contact with the breast and an outer portion which adheres effectively to maintain the pad in position during non-nursing times.

### FIELD OF THE INVENTION

Breast pads are used by lactating women to prevent milk from leaking from the breast and to prevent staining of clothing and linen when not nursing. The pad is generally made of a multi-layer material with high absorbency and is concave in shape so as to directly fit over the breast as best as possible. During the term of nursing, production of the milk continues without regard to the time of day or the feeding schedule of the child.

Milk production can cause leaking from the nipples, and especially at night, in the absence of positioning the pad over the nipple to absorb the milk and avoid staining of clothing. Therefore, high absorption by these pads is indispensable. But, since a milk-saturated pad would remain in contact with the breast, disposable pads that permitted frequent changing came into existence.

Even so, it become desirous to securely maintain the breast pad in a fixed position over the breast, thus giving rise to the advent of adhesive materials applied to the outer surface of the pad to adhere it to the inner surface of a nursing brassiere, and this is especially the case when attempting to keep the breast pad in the correct position during sleep when leaking is more likely to stain bed clothing and materials.

To address this problem, nursing pads are available that use an adhesive on an outer surface, but these pads are still of limited use because of the area of the application of the adhesive material on the pad. As such, nursing breast pads are easily detachable from a women's garment and thereby cause removal of the pad from the women's nipple or folding over of the pad on itself to defeat the purpose or objective of the pad in the first instance.

U.S. Pat. No. 7,008,409 disclose a disposable nursing breast pad contoured to fit the breast of a nursing woman, and comprised of a soft, highly absorbent material. Disposed adjacent the center of the pad's inner surface is a lubricating moisturizer, to protect the woman's nipple and prevent dryness and cracking. The pad's outer surface is provided with an adhesive for adhering to the inner surface of clothing, such as a nursing brassiere, to maintain the nursing pad in position on the woman's nipple when not nursing, and to absorb leaked milk and prevent staining of a garment. The adhesive is in the form of a pair of arcuate shaped strips disposed above and below the center of the pad and adjacent to the pad's outer periphery. See FIG. 2.

A bra for nursing mothers comprising housing B on the inside of the bra (shown in FIG. 1) to hold an absorbent disposable pad against the breast of the mother to absorb any secreted liquid and a waterproof lining A to the cups of the bra to prevent the liquid from leaking through the bra onto the clothing of the mother is disclosed in GB 2429898.

U.S. Pat. No. 5,149,336 disclose a disposable, contoured, form fitting, absorbent breast pad comprising three layers, an inner fluid wicking layer, a central fluid absorbent layer, and an outer moisture resistant layer, tear drop in shape and dome-like in cross-section, and is large enough to encompass substantially the entire female breast and to fill substantially the average nursing and maternity bra cup. See FIGS. 3 and 4 which disclose the breast pads, and FIG. 5 which shows how it is disposed within the brassiere.

A disposable nursing pad, worn again after breast-feeding, which prevents the nursing pad from losing its shape during breast-feeding is disclosed in published U.S. application No. 2007/0099542. The nursing pad includes a nursing pad main body 10 including a first folding portion arranged such that it passes a portion near the apex of the nursing pad main body, and extends up to a portion around the perimeter, and which serves as a folding axis along which the nursing pad 10 can be folded. The nursing pad further includes a wrapping member or an instruction manual that provides the information which instructs the user to wear the nursing pad main body with the first folding portion 15, which is positioned obliquely from the upper-outer side to the lower-inner side of the user's breast. The nursing pad with this a structure allows the user to wear the nursing pad again after breast-feeding.

There is need in the art of disposable nursing breast pads to maintain the nursing breast pad firmly fixed in position over the breast, and particularly the nipple.

There is further need in the art of disposable nursing breast pads to have the outer surface of the nursing breast pad firmly fixed to an inner surface of the worn garment—such as a nursing brassiere, as it is especially challenging to keep the nursing breast pad in a fixed position at night, when asleep, when leaking is more highly likely to occur and soil bed clothing and materials.

A still further need in the art of disposable nursing breast pads is to utilize an adhesive in a manner which safely engages the skin but also effectively serves to prevent the pad from leaking and rotating, twisting, bending, creasing or bulging out of place and thereby avoid the unwanted hassle of repositioning the pad continuously or having to replace the worn pad with a new one.

### SUMMARY OF THE INVENTION

One object of the present invention is to provide a disposable nursing breast pad that maintains the nursing breast pad firmly fixed in position over the breast, and particularly the nipple part of the breast.

Another object of the invention is to provide a disposable nursing breast pad having the outer surface of the breast pad firmly fixed to an inner surface of the worn garment, such as a nursing brassiere, to resolve the problem of keeping the nursing breast pad in fixed position at night when asleep, when leaking is more likely to occur and soil clothing and bedding materials.

A still further object of the present invention is to provide a disposable nursing breast pad comprising an adhesive which safely engages the skin but also effectively serves to prevent the pad from leaking and rotating, twisting, bending, creasing or bulging out of place, so as to avoid the unwanted hassle of repositioning the pad continuously or having to replace the worn pad with a new one.

These and other objects of the invention will become more apparent by reference to the drawings and specification.

### BRIEF DESCRIPTION OF THE DRAWINGS

FIG. 1 is a view in perspective showing an innermost section of a disposable nursing pad of the invention, which, when the multi-functioning pressure sensitive adhesive portion is positioned in contact with the breast both absorbs milk that has leaked and simultaneously lubricates or medicates the nipple.

FIG. 2 is a view in perspective of an outermost section of a different embodiment of the disposable nursing pad in accordance with the invention, showing the multi-functioning pressure sensitive adhesive composition as an all-around adhesive backing to provide stability to the pad and to secure in place the inner surface of clothing, such as a regular nursing brassier or blouse.

### DETAILED DESCRIPTION OF PREFERRED EMBODIMENTS

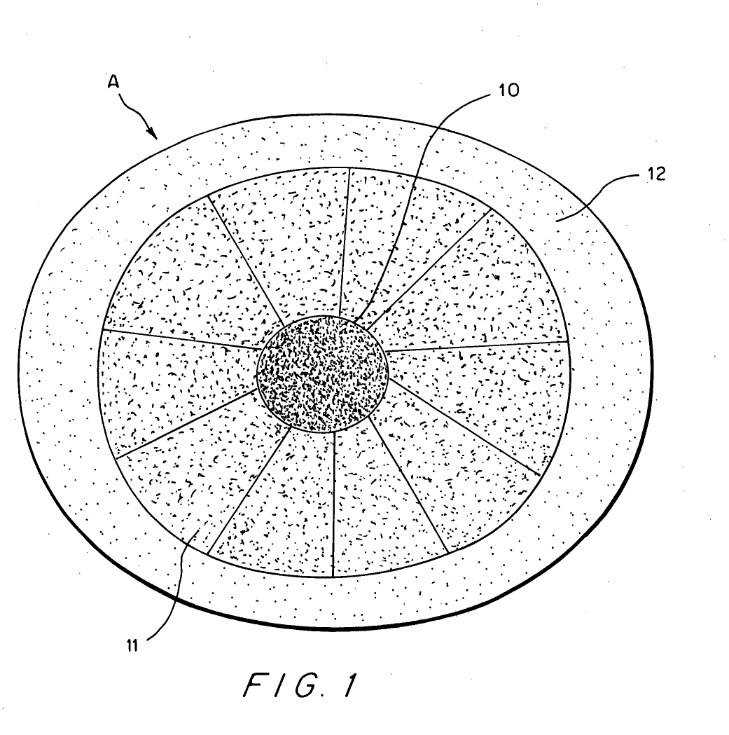

In the context of the invention, reference is now made to FIG. 1 which shows a view in perspective of an innermost section of a disposable nursing pad A, wherein the innermost section is a multi-functioning, pressure sensitive adhesive that is placed in contact with the breast to absorb leaking milk while simultaneously lubricating or soothing a woman's nipple. The multi-functioning, pressure sensitive adhesive layer comprises one or more poly isobutylenes elastomers, and one or more moisture absorbing moisture transmitting, water soluble and/or water swellable agents.

More particularly, the pressure sensitive multi-functioning pressure sensitive adhesive may contain a pharmaceutically active ingredient, and the adhesive layer is capable of releasing the active ingredient to the surface of the skin over a period of time of from several hours to one or more days.

The a multi-functioning, pressure sensitive adhesive comprises a homogeneous blend of one or more polyisobutylenes or a mixture of one or more polyisobutylenes and an elastomer such as butyl rubber and/or styrene radial or block type copolymers and one or more moisture absorbing, moisture transmitting, water soluble or water swellable agents. The adhesive may also contain other ingredients such as mineral oil and lubricants.

The pharmaceutically active ingredient incorporated into the adhesive layer is at the skin-contacting surface and in the strata of the adhesive layer closest to the skin-contacting surface. This is accomplished by forming a suspension of the active ingredient in a material which is compatible with the composition of the adhesive layer, and this suspension is cast onto a sheet of silicone coated release paper which is laminated to the skin contacting surface of the adhesive layer.

The pressure sensitive adhesive dressing consists of an adhesive layer formulated from materials suitable for use on human skin and a flexible backing layer. The adhesive layer must be capable of bonding to the skin for from several hours to several days.

Suitable pressure sensitive adhesive compositions for use as the skin contacting multi-functioning adhesive layer are homogeneous blends of one or more polyisobutylenes or mixtures of one or more polyisobutylenes and an elastomer such as butyl rubber, medium or high molecular weight polyisobutylene and/or styrene radial or block type copolymers and one or more moisture absorbing, moisture transmitting, water soluble and/or water swellable agents. The adhesive compositions may contain other optional ingredients such as tackifiers and antioxidants.

The polyisobutylene component of the pressure sensitive adhesive compositions function to provide adhesion to dry body surfaces, i.e., dry tack, and along with the flexible backing layer or tab (not shown) maintain structural integrity until the tab or backing is removed immediately before applying to the breast. Preferably, the polyisobutylenes are one or more low molecular weight polyisobutylenes having a viscosity average molecular weight of from about 8,700 to about 1,700 (Staudinger).

One or more elastomers may be combined with the polyisobutylene, and these elastomer materials function to increase the elasticity, tear resistance, and cohesiveness of the adhesive compositions. Suitable elastomers include butyl rubber which is a copolymer of isobutylene with a minor amount of isoprene having a viscosity average molecular weight of from about 350,000 to about 50,000 (Florey), medium and high molecular weight polyisobutylene having a viscosity average molecular weight of from about 81,000 to about 99,000 (Staudinger) and styrene radial or block copolymers. Especially suitable styrene copolymers include styrene-butadiene-styrene (S-B-S) and styrene-isoprene-styrene (S-I-S) block type copolymers both of which are commercially available.

Significantly, the moisture absorbing, moisture transmitting, water soluble or water swellable agents permit the adhesive compositions to adhere to moist body surfaces, i.e., wet tack. Additionally, the absorption of moisture and milk from the surface of the skin into the adhesive layer results in a microenvironment at the adhesive layer-skin interface caused by hydration of these agents at the adhesive surface and gradually throughout the entire adhesive layers. This results in the continuous release of the topically active pharmaceutical agent from the adhesive layer. Suitable water soluble agents for incorporation within the adhesive compositions are hydrocolloid gums including sodium carboxymethylcellulose, potassium carboxymethylcellulose, pectin, gelatin, guar gum, locust bean gum, gum karaya, xanthan gum, mixed sodium/calcium salts of alginic acid, carrageenan, etc., and mixtures thereof include substantially water insoluble cross-linked sodium carboxymethylcellulose.

Mineral oil can be included within the adhesive layer both as a component of the pressure sensitive adhesive formulation and, as the preferred agent for incorporating or depositing the pharmaceutically active agent at the skin contacting surface and in the strata of the adhesive layer closest to the skin contacting surface. Tackifiers can also be included within the adhesive layer. Suitable tackifiers include the pentaerythritol esters.

The thickness of the adhesive layer can be varied. In general, the adhesive layer will be from about 20 to about 100 mils. Thick nesses greater than 100 mils are undesirable as being uncomfortable to wear and more susceptible to accidental removal to destabilize or loosen the adhesive pad and cause leaking and soiling of the nursing brassiere and garments.

Using the adhesive structure of the invention avoids the necessity of having to deposit medication on a separate section of the breast pad 10 and yet another section of the breast pad normally utilized as the adhesive section.

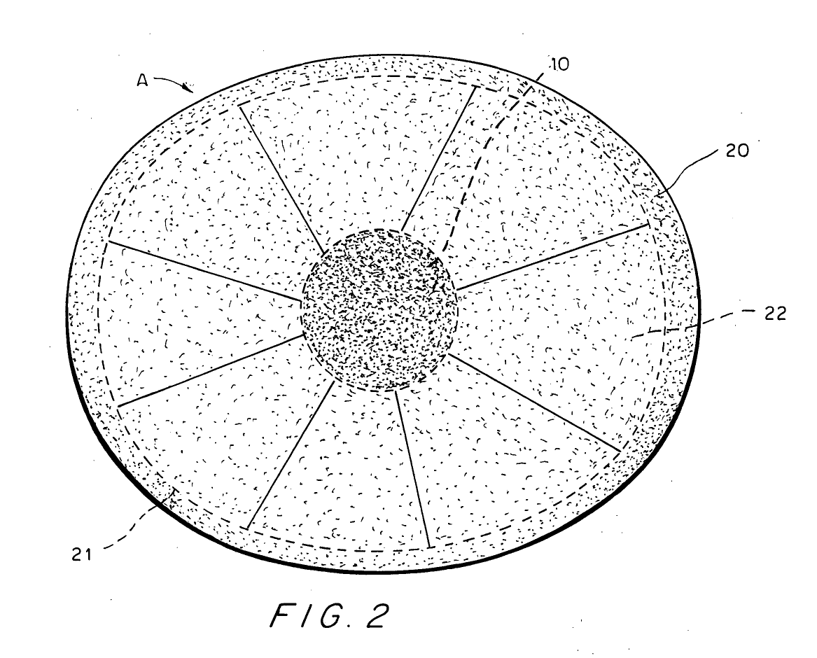

As can be seen from FIG. 1, the disposable nursing pad A is circular in shape and/or contoured so as to cover a woman's breast completely. Further, the innermost section of the nursing breast pad A is concave in shape, in contrast to the outer section, as depicted in FIG. 2, which is convex in shape. The nursing breast pad A is a multi-layer structure having an inner absorbent layer 11 in contact with the breast and an outer peripheral layer 12. The inner absorbent section or layer 11 and the outer peripheral layer 12 may be bonded together by thermal bonding or other conventional means, around nipple pad 10. While the inner absorbent layer may be comprised of a fabric material that is high in fluid absorptiveness, the outer layer is composed of a moisture impermeable material to accommodate the fact that it is positioned directly against the clothing of a woman, such as the nursing brassier (not depicted).

With regard to FIG. 2, there is shown a view in perspective of the outer, concave sectional portion of another embodiment of the disposable nursing breast pad A, wherein the breast pad A includes an outer peripheral circumference 20, and an inner layer 21 inscribed within the peripheral circumference. This nursing breast pad A has the multi-functioning adhesive 22, disposed throughout all of the inner layer, which may extend to the border of the outer peripheral circumferential layer to provide assurance against slippage or dislodging of the nursing breast pad from a fixed position that prevents milk from leaking and soiling the brassier, clothing or linen. This embodiment of the nursing breast pad with its concave surface disposed in a facing relationship to the center portion of the nursing breast pad ensures firm gripping action of the multi-functioning adhesive in prevention of distortion of the nursing breast pad. As can be seen, the adhesive section in FIG. 2 is disposed adjacent the edges of the pads outer layer 20 to afford more formidable adherence and secure holding of the nursing breast pad over the breast. Further, since the multi-functional adhesive layer is moisture transmitting, lubricant transmitting, and pharmaceutically, active ingredient transmitting, these transmitted materials are naturally transmitted to the nipple beneath the pad itself.

While the invention has been shown in its preferred embodiments, it will be apparent to those skilled in the art that numerous changes and modifications may be made without departing from the scope of the invention, which is described by way of illustration and not limitation, in that the actual scope of the invention is defined in the claims hereafter presented, in relation to existing prior art.

I claim:

1. A disposable breast pad containing a multi-functional pressure sensitive adhesive to hold said breast pad and adjoining bra firmly in place without slippage upon leakage of fluids from the breast of nursing or pregnant human females, comprising:
a) outer peripheral circumference layer means disposed on a women's breast;
b) inner absorbent layer means disposed on an inner section of said outer layer means in contact with the breast to absorb leaking secreted fluids;
wherein said inner absorbent layer is a multi-functional pressure sensitive adhesive that is moisture absorbing, and moisture transmitting.

2. The disposable breast pad of claim 1, wherein said multi-functional pressure sensitive adhesive further contains a water swellable agent.

3. The disposable breast pad of claim 1, wherein said multi-functional pressure sensitive adhesive further contains a water swellable agent.

4. The disposable breast pad of claim 1, wherein said multi-functional pressure sensitive adhesive further contains a pharmaceutically active ingredient.

5. The disposable breast pad of claim 1, wherein said multi-functional pressure sensitive adhesive further contains a lubricant,

6. The disposable breast pad of claim 5, wherein said lubricant is mineral oil.

7. The disposable breast pad of claim 1, wherein said pressure sensitive adhesive comprises a homogeneous blend of one or more polyisobutylenes or a mixture of one or more polyisobutylenes and an elastomer.

8. The disposable breast pad of claim 7, wherein said elastomer is butyl rubber and or/ styrene radial or block type copolymers.

9. The disposable breast pad of claim 1, wherein said inner absorbent layer means extends to the border of said outer peripheral circumference layer means.
